(19) United States
(12) Patent Application Publication
(10) Pub. No.: US 2023/0031009 A1
(43) Pub. Date: Feb. 2, 2023

(54) TREATMENT FOR CHEMOBRAIN

(71) Applicant: YALE UNIVERSITY, NEW HAVEN, CT (US)

(72) Inventors: Barbara Ehrlich, New Haven, CT (US); Lien Nguyen, Jamaica Plain, MA (US)

(21) Appl. No.: 17/801,486

(22) PCT Filed: Feb. 11, 2021
(86) PCT No.: PCT/US2021/017661

§ 371 Date: Aug. 22, 2022

Related U.S. Application Data
(60) Provisional application No. 63/028,976, filed on May 22, 2020.
(60) Provisional application No. 62/980,718, filed on Feb. 24, 2020.

(51) Int. Cl. A61K 31/4741; A61K 31/337; A61K 31/437; A61K 31/675; A61K 31/704; A61P 25/28; A61K 33/14

(52) U.S. Cl. CPC A61K 31/4741; A61K 31/337; A61K 31/437; A61K 31/675; A61K 31/704; A61P 25/28; A61K 33/14

(57) ABSTRACT

The present invention is directed to a method of treatment, including prevention or reducing the likelihood of adverse effects of chemotherapy comprising reducing and/or inhibiting chemotherapy-induced adverse effects (CIAE), especially central nervous system adverse effects, such as cognitive effects (especially chemotherapy induced cognitive impairment or CICI, also referred to as reduced cognition, cognitive impairment or chemobrain) by administering to the patient in need, including co-administering to the subject in need a pharmaceutically effective amount of a protein kinase C (PKC, often, PKC α and/or β) inhibitor, alone or in combination with a lithium salt. Related pharmaceutical compositions are also provided by the present invention.

### CLAIM OF PRIORITY

This application claims the benefit of priority of U.S. provisional application Ser. No. 62/980,718, filed 24 Feb. 2020, and U.S. 63/028,976, filed 22 May 2020 the entire contents of which applications are incorporated by reference herein.

### GOVERNMENT INTEREST

The invention in this patent application was made with government support under Grant No. 5P01DK057751 awarded by the National Institutes of Health. The Government has certain rights in the invention.

### FIELD OF THE INVENTION

The present invention is directed to a method of treatment, including prevention or reducing the likelihood of effects comprising reducing and/or inhibiting chemotherapy-induced adverse effects (CIAE), especially central nervous system adverse effects, such as cognitive effects (especially chemotherapy induced cognitive impairment or CICI, also referred to as reduced cognition, cognitive impairment or chemobrain) by administering to the patient in need, including co-administering to the subject in need a pharmaceutically effective amount of a protein kinase C (PKC, often, PKC α and/or β) inhibitor, alone or in combination with a lithium salt. Related pharmaceutical compositions are also provided by the present invention.

### BACKGROUND OF THE INVENTION

Approximately 40% of cancer survivors receiving chemotherapy suffer from cognitive impairment, including memory lapses, learning difficulties, and attention deficits [1]. However, the specific cognitive functions affected, the trajectories of cognitive changes, and the underlying mechanisms remain unclear. Recent evidence from structural [2, 3] and functional [4-6] imaging studies on human patients, as well as experiments using animal models, show that chemotherapy-induced cognitive impairment (CICI, also know as chemobrain) is a serious side effect that persists many years after treatment ends. Because the timing of the neurological insult, which is the beginning of chemotherapy, is known, determining the cellular and molecular mechanisms of chemobrain allows the discovery process for better prevention and treatment options.

### BRIEF DESCRIPTION OF THE INVENTION

Using the inventors' extensive knowledge of the calcium signaling complex that includes neuronal calcium sensor 1 (NCS1), the inventors have identified drugs that stabilize cell survival and function in the context of chemobrain, both reducing the likelihood of chemobrain as well as inhibiting chemobrain (especially cognition impairment and memory loss, both short-term and long-term) in instances where such conditions exist secondary to chemotherapy, especially with a taxane (especially paclitaxel or docetaxel), cyclophosphamide, doxorubicin (Adriamycin), 5-fluorouracail (5-FU) or a vinca alkaloid as otherwise disclosed herein. The inventors unexpectedly discovered that memory deficits and/or cognitive impairment which occur in a patient secondary to chemotherapy treatment could be rescued through pre-treatment, co-administration or therapy of chemobrain with a PKC inhibitor, especially including chelerythrine chloride. The present invention thus predicts PKC inhibitors alone or PKC inhibitors in combination with a lithium salt as therapeutic agents to prevent and/or treat chemotherapy-induced cognitive impairment (chemobrain). PKC inhibitors and in particular chelrythrine, or a pharmaceutically acceptable salt thereof have never been considered for treatment and/or prevention of chemobrain. In addition, the use of at least one PKC inhibitor alone in combination with a lithium salt have shown unexpected activity, including as synergistic agents in the treatment and/or prevention of chemobrain.

Pursuant to the present invention, an effective amount of a least one PKC inhibitor, for example chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432, ruboxistaurin, miyabenol C, myricitrin, gossypol, verbascoside, BIM-1, staurosporine, sotrastaurine, bryostatin 1, Gouml 6983, Go 6976, Ro 32-0432, enzastaurin (LY317615), GSK690693, fasudil (JA-1077), mitoxantrone, bisindolylmaleimide (GF109203X), RO31-8220, rotterin, K252a, baicalein, quercetin, luteolin, bisindolylmaleimide II, calphostin C, L-threo-dihydrosphingosine, D-erythro-sphogosine, melittin, midostaurin (PKC412), CGP 533353, CRT 0066864, (±)-palmitoylcarnitine, PKC412, PKCβ pseudosubstrate, PKCζ pseudosubstrate, z-pseudosubstrate inhibitory peptide (ZIP) or a pharmaceutical salt or mixture thereof, among others as described herein or often at least one PKC inhibitor which is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432 hydrochloride salt or a pharmaceutical salt, most often chelerythine or a pharmaceutically acceptable salt is pre-administered to a patient or subject at least one hour to up to 10 days (often, at least an hour to a day or two) before therapy with a chemotherapeutic agent which causes chemobrain, co-administered at approximately the same time, including concurrently and/or sequentially with the chemotherapeutic agent or administered post chemotherapy (from approximately an hour to up to 10 days, preferably from an hour up to about 24 hours post-therapy) in order to reduce the likelihood, inhibit and/or treat cognitive deficits including memory loss and cognitive impairment associated with chemobrain. In embodiments, the chemotherapy agents include taxanes, vinca alkaloids, cyclophosphamide, adriamycin and 5-fluorouracil, or pharmaceutically acceptable salts thereof. In embodiments, the administration of the PKC inhibitor also includes pre-administration, co-administration or post-administration of chemotherapy treatment with an effective amount of a lithium salt to reduce the likelihood, inhibit and/or treat the cognitive defects (including memory loss and cognitive impairment) associated with the administration of the chemotherapeutic agent. In embodiments, the PKC inhibitor and lithium salt are both administered pre-treatment, at approximately the same time as or shortly after the chemotherapy treatment. In preferred embodiments directed to methods or compositions according to the present invention, the chemotherapy agent is often a taxane, often paclitaxel or docetaxel, the PKC inhibitor is chelerythrine or a pharmaceutically acceptable salt thereof and the lithium salt is lithium carbonate or lithium chloride, more often lithium chloride.

In embodiments, the present invention is directed to pharmaceutical compositions comprising an effective amount of at least one PKC inhibitor as described herein, in combination with at least one lithium salt (e.g., lithium chloride, lithium carbonate, lithium acetate, lithium sulfate, lithium citrate, lithium orotate and lithium gluconate, preferably lithium chloride or lithium carbonate, more often lithium chloride), in combination with a pharmaceutically acceptable carrier, additive and/or excipient for the prevention, inhibition and/or treatment of chemobrain secondary to chemotherapy treatment with one or more of a taxane, a vinca alkaloid, cyclophosphamide, doxorubicin (Adriamycin), 5-fluorouracil or a pharmaceutically acceptable salt thereof.

In still other embodiments, the invention is directed to and provides a pharmaceutical composition comprising:
(a) a pharmaceutically-effective amount of one or more anti-cancer active ingredients selected from the group consisting of a taxane, a vinca alkaloid, cyclophosphamide, doxorubicin (Adriamycin), 5-fluorouracil, a pharmaceutically acceptable salt or a mixture thereof;
(b) one or more PKC inhibitors (e.g. chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432 hydrochloride salt or a pharmaceutical salt or mixture thereof); optionally in combination with at least one lithium salt (e.g. lithium chloride, lithium carbonate, lithium acetate, lithium sulfate, lithium citrate, lithium orotate and lithium gluconate, preferably lithium chloride or lithium carbonate); and
(c) a pharmaceutically-acceptable carrier, additive and/or excipient. In embodiments, the pharmaceutical compositions comprises one or more anti-cancer active ingredient as described above, one or more PKC inhibitor, one or more lithium salt and a pharmaceutically acceptable carrier additive and/or excipient, all components being included in effective amounts.

In a preferred embodiment, the invention provides a method of treatment comprising reducing the likelihood of, reducing/reversing and/or inhibiting chemotherapy-induced adverse effects (CIAE) including central nervous system adverse effects, such as cognitive effects (especially reduced cognition, cognitive impairment and memory loss) in a subject who suffers from cancer and is treated with one or more of a taxane, such as Paclitaxel (Taxol®), docetaxel (Taxoterel®) or other taxane, a vinca alkaloid such as vincristine, vinblastine, etc., cyclophosphamide, adriamycin or 5-fluorouracil by pre-administering, co-administering or post-administering to the subject a pharmaceutically effective amount of a PKC inhibitor, optionally in combination with a lithium salt as described herein.

In embodiments, the PKC inhibitor, alone or in combination with a lithium salt is co-administered to the subject prior to, contemporaneously with, or after administration of the CIAE-inducing anti-cancer active ingredient (chemotherapeutic agent). In alternative embodiments, the PKC inhibitor is administered in combination with a lithium salt at different times prior to, contemporaneously with or after administration of the CIAE-inducing chemotherapeutic agent. Often, at least one PKC inhibitor or at least one PKC inhibitor in combination with at least one lithium salt is administered to the subject prior to the administration of the chemotherapeutic agent (CIAE-inducing anti-cancer active ingredient) (e.g. around one hour to several days or more, often four hours or more, or around three hours, or around two hours, or around one hour or less as otherwise described herein before administration of the CIAE-inducing anti-cancer active ingredient(s)).

These and/or other aspects of the invention are readily gleaned from a review of the detailed description of the invention which follows.

### BRIEF DESCRIPTION OF THE FIGURES

FIG. 1 shows that lithium pre-treatment rescues paclitaxel-induced short-term memory impairment (A) Schematic illustration of paclitaxel and lithium injection scheme, followed by behavioral tasks (OF=open-field exploration, DOR=displaced object recognition, D=displaced, F=familiar). (B-D) Locomotor activity and anxiety were similar among all groups at 4 DPI and 22 DPI (one-way ANOVA, p>0.3 for all comparisons). (F) At 5 DPI, mice that received saline or lithium followed by vehicle injection showed significant preference for the displaced object (t-test, adjusted for multiple comparisons, p=0.004 and p=0.003). Mice that received saline and paclitaxel showed no discrimination between the two objects (p=0.4). Mice that received lithium and paclitaxel showed significant discrimination (p=0.002). (G) No significant differences were observed for total interaction time (one-way ANOVA, p=0.4). (H-I) Similar results were obtained at 23 DPI. n=10-17 mice per group.

FIG. 2 shows that Lithium post-treatment within a limited window reverses paclitaxel-induced short-term memory impairment (A) Schematic illustration of paclitaxel and lithium injection scheme, followed by behavioral tasks (OF=open-field exploration, DOR=displaced object recognition, D=displaced, F=familiar). (B) At 5 DPI, group 1 showed significant preference for the displaced object (p=0.001). Groups 2 and 3 showed no discrimination between the two objects (p=0.6 and p=0.8, respectively). (C) At 23 DPI, groups 1 and 2 showed significant preference for the displaced object (p=0.02 and p=0.003, respectively). Group 3 showed no discrimination between the two objects (p=0.99), (D) While groups 1 and 3 were consistent in their choice preference between the two sessions, group 2 showed a significant trend from no preference to preference for the displaced object (paired t-test, p=0.03). n=6-7 mice per group.

FIG. 3 shows Golgi-Cox staining and quantification of granule cells in the dentate gyrus of the hippocampus 30 DPI. (A) Schematic diagram showing the region of the coronal section where the cells were imaged (B) Representative images of granule cells from each group. (C) Sholl analysis revealed a substantial reduction in dendritic complexity in the group receiving saline and paclitaxel, particularly from 20 to 90 μm from the soma (repeated measures two-way ANOVA, followed by Dunnett's test). Lithium pre-treatment rescued the reduction to the level comparable to those of the two groups receiving vehicle control. (D) Similarly, compared to other groups, granule cells from the group treated with saline and paclitaxel showed a significant reduction in total (?) dendritic length (One-way ANOVA, followed by Tukey post-hoc test). n=12 to 16 neurons total from 4-6 mice per group. Non-linear 6^{th} order polynomial fit was used for C.

FIG. 4 shows Golgi-Cox staining and quantification of layers 2/3 cortical pyramidal neurons in the frontal cortex 30 DPI. (A) Schematic diagram showing the region in the coronal section where the cells were imaged. (B) Representative images of a cortical pyramidal cell. (C) Sholl analysis revealed a significant reduction in dendritic complexity in the group receiving saline and paclitaxel (repeated measures two-way ANOVA). Lithium pre-treatment rescued the reduction to the level comparable to those of the two groups receiving vehicle control. (D) Compared to other groups, cortical neurons from the group treated with saline and paclitaxel showed a significant reduction in total dendritic length (One-way ANOVA, followed by Tukey's post-hoc test). (E-F) Further analysis revealed that the reduction in complexity in length was not observed in basal dendrites (p-treatment=0.57) but (G-H) was primarily due to differences in apical dendrites (p-treatment=0.038). n=12 to 16 neurons total from 4-6 mice per group. Non-linear 3^{rd} order polynomial fit was used for C, E, and, G.

FIG. 5 shows the upregulation of PKC and pMARCKS in the hippocampus and the cortex.

FIG. 6 shows PKC inhibitor Chelerythrine (Chel) pre-treatment rescues paclitaxel-induced short-term memory impairment (A) Schematic illustration of paclitaxel and Chel injection, followed by behavioral tasks (OF=open-field exploration, DOR=displaced object recognition, D=displaced, F=familiar). (B-D) Locomotor activity and anxiety were similar among all groups at 4 DPI and 22 DPI (one-way ANOVA, p>0.4 for all comparisons). (F) At 5 DPI, mice that received saline or Chel followed by vehicle injection showed significant preference for the displaced object (t-test, adjusted for multiple comparisons, p=0.03 and p=0.005). Mice that received saline and paclitaxel showed no discrimination between the two objects (p=0.75). Mice that received lithium and paclitaxel showed significant discrimination (p=0.005). (G) No significant differences were observed for total interaction time (one-way ANOVA, p=0.86), (H-I) Similar results were obtained at 23 DPI. n=5-8 mice per group.

FIG. 7 shows a proposed model for the mechanism of paclitaxel-induced cognitive impairment. Paclitaxel binding to NCS1 enhanced NCS1 binding to the InsP3R, resulted in an increase in calcium release from the ER into the cytoplasm. The increase in calcium concentration, as well as an upregulation of PKCα, results in PKC hyperactivity. PKCα in turns phosphorylates MARCKS into p-MARCKS, leading to actin instability. This in turn leads to loss of dendrites in the hippocampus and frontal cortex, and hence cognitive impairment. Lithium, through inhibiting InsP3R-dependent calcium release and PKCα, and Chel, through inhibiting PKCα, can rescue paclitaxel-induced cognitive impairment.

FIG. S1 shows the optimization of paclitaxel injection and lithium pretreatment. (A) Schematic illustration for paclitaxel and lithium injection, followed by behavioral tasks (OF=open-field exploration, DOR=displaced object recognition, n=5 mice per group). (B) Weight was measured daily before and after paclitaxel injection and normalized to the first day of injection. The red triangles indicated days with paclitaxel injection. Mice lost approximately 5-10% of their body weights after 2 injections but quickly recovered afterward. (C-D) At 5 and 23 DPI, paclitaxel-only mice did not discriminate between the objects (p=0.54 and p=0.48 respectively). Mice receiving both paclitaxel and 4×12.8 mg/kg LiCl spent significantly more time exploring the displaced object compared to the familiar object on both days (both p<0.005). Mice receiving both paclitaxel and 8×12.8 mg/kg LiCl or 4×25.6 mg/kg LiCl showed mixed results. N=5 mice per group.

FIG. S2 show that an efficacious dose of lithium is below the common therapeutic range. Mouse plasma lithium level following a 12.8 mg/kg intraperitoneal injection of lithium. Plasma lithium peaked at 0.36 mM, which is below the lower therapeutic target range (0.5 to 0.8 mM) in humans. Lithium is almost cleared out from the system 6 hours after injection. N=34 mice for each time point. Blood samples were obtained through cardiac puncture. Lithium concentration was measure by Yale Laboratory Medicine using a colorimetric assay.

FIG. S3 shows that weights are not different among the 4 groups. Weight was measured daily before and after paclitaxel injection and normalized to the first day of injection. The red triangles indicated days with paclitaxel injection. Mice lost approximately 5% of their body weights during injections but quickly recovered afterward. No significant differences among groups were found (mixed ANOVA with correction of repeated measures, group factor=0.08). N=10-17 mice per group.

### DETAILED DESCRIPTION OF THE INVENTION

In accordance with the present invention there may be employed conventional cell culture methods, chemical synthetic methods and other biological and pharmaceutical techniques within the skill of the art. Such techniques are well-known and are otherwise explained fully in the literature.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It is to be noted that as used herein and in the appended claims, the singular forms “a,” “and” and “the” include plural references unless the context clearly dictates otherwise.

The following terms, among others, are used to describe the present invention. It is to be understood that a term which is not specifically defined is to be given a meaning consistent with the use of that term within the context of the present invention as understood by those of ordinary skill.

The term “compound” or “agent”, as used herein, unless otherwise indicated, refers to any specific chemical compound disclosed herein and includes tautomers, regioisomers, geometric isomers as applicable, and also where applicable, optical isomers (e.g. enantiomers) thereof, as well as pharmaceutically acceptable salts (such that the PKC inhibitor or lithium or another agent refers to that agent where applicable and any pharmaceutically acceptable salt) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal. In the present invention, the patient or subject referred to is often a human cancer patient.

The terms “effective” or “pharmaceutically effective” are used herein, unless otherwise indicated, to describe an amount of a compound or composition which, in context, is used to produce or affect an intended result, whether that result relates to the inhibition of the effects of CIAE, especially cognitive impairment or chemobrain (including short-term and long-term memory loss), or to potentiate the effects of a concomitant treatment of cancer. This term subsumes all other effective amount or effective concentration terms (including the term “therapeutically effective”) which are otherwise described in the present application.

The terms “treat”, “treating”, and “treatment”, etc., as used herein, refer to any action providing a benefit to a patient at risk for or afflicted by a cancer and who suffers from or is at risk of developing CIAE, especially central nervous system effects such as cognitive impairment or chemobrain. Treatment, as used herein, encompasses both prophylactic (reducing the likelihood of an adverse effect occurring) and therapeutic treatment (inhibiting or even reversing the adverse effect). Favorable or successful treatment of chemobrain includes ameliorating, reducing the effect, or inhibiting and/or reversing one or more symptoms of chemobrain, including confusion, impaired concentration, difficulty finding the right word, being unusually disorganized, difficulty learning new skills, difficulty multitasking, feeling of mental fogginess, short attention span, short-term and long-term memory problems, taking longer to complete routine tasks, impaired verbal memory, such as remembering a conversation and trouble with visual memory, such as recalling an image or list of words. Favorable prophylaxis includes reducing the likelihood and/or preventing in certain patients or subjects one or more of the symptoms of chemobrain by pre-treating or co-administering a PKC inhibitor and optional lithium salt prior to or during anticancer therapy.

The term “therapy induced side effects” including “chemotherapy induced adverse effects” or “CIAE” refers to adverse side effects which occur secondary to the administration of certain types of therapeutic agents, especially including chemotherapy, in particular, the taxanes, the vinca alkaloids, cyclophosphamide, Adriamycin and 5-fluorouracil as otherwise described herein pursuant to the treatment of cancer in a patient. The terms chemotherapy and cancer therapy may be used synonymously within context herein and the term therapy subsumes chemotherapy and cancer therapy. The term CIAE includes central nervous system impairment, especially cognitive impairment, chemobrain, foggy thought process and short-term and long-term memory loss. This cognitive impairment is often referred to as chemobrain.

Symptoms of chemotherapy induced cognitive impairment (CICI) or chemobrain include, but are not limited to, thinking and cognitive problems including foggy thought process, cognitive impairment, lack of/diminished concentration and short- and long-term memory loss. The symptoms associated with chemobrain include confusion, impaired concentration, difficulty finding the right word, being unusually disorganized, difficulty teaming new skills, difficulty multitasking, feeling of mental fogginess, short attention span, short-term and long-term memory problems, taking longer to complete routine tasks, impaired verbal memory, such as remembering a conversation and trouble with visual memory, such as recalling an image or list of words.

The term “Protein kinase C or “PKC” is used to refer to protein kinase C, which is a family of protein kinase enzymes that are involved in controlling the function of other proteins through phosphorylation mechanisms and play important roles in several signal transduction cascades. The PKC family consists of fifteen isozymes in humans. In the present invention, the isozymes of PKC which are involved in cognitive impairment associated chemotherapy induced adverse effects include PKCα, β1 and β2. In the present invention, the teen PKC inhibitor refers to an inhibitor of PKCα, PKCβ1 and/or PKCβ2. Exemplary PKC inhibitors include for example, chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432, ruboxistaurin, miyabenol C, myricitrin, gossypol, verbascoside, BIM-1, staurosporine, sotrastaurine, bryostatin 1, Gouml 6983, Go 6976, Ro 32-0432, enzastaurin (LY317615), GSK690693, fasudil (JA-1077), mitoxantrone, bisindolylmaleimide (GF109203X), RO31-8220, rotterin, K252a, baicalein, quercetin, luteolin, bisindolylmaleimide II, calphostin C, L-threo-dihydrosphingosine, D-erythro-sphogosine, melittin, midostaurin (PKC412), CGP 533353, CRT 0066864, (±)-palmitoylcarnitine, PKC412, PKCβ pseudosubstrate, PKCζ pseudosubstrate, z-pseudosubstrate inhibitory peptide (ZIP) or a pharmaceutical salt or mixture thereof. Preferred PKC inhibitors in the present invention include chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432 hydrochloride salt or a pharmaceutical salt or mixture thereof. A particularly useful PKC inhibitor is chelerythrine or a pharmaceutically acceptable salt thereof.

“Taxanes” as used herein include, but are not limited to, paclitaxel (Taxol®), docetaxel (Taxoterel®), taxane derivatives such as IDN 5390, GRN1005, the taxane derivatives described in EP 2330100A1, and the taxane derivatives described or referenced in Bioscience, Biotechnology, and Biochemistry, Vol. 76 (2012), No. 2 pp. 349-352.

“Vinca alkaloids” include, but are not limited to, vinblastine, vincristine, vindesine and vinorelbine and the vinca alkaloids described or referenced in Holland-Frei Cancer Medicine. 6th edition, Kufe D W, Pollock R E, Weichselbaum R R, et al., editors. Hamilton (ON): BC Decker; 2003.

The taxanes, vinca alkaloids, cyclophosphamide, Adriamycin and 5-fluorouracil among other agents, belong to a group of compounds or agents referred to as CIAE-inducing anti-cancer active ingredients that during therapy, cause chemobrain and are associated with cognitive impairment, memory loss (both short-term and long-term), loss of attention and foggy brain which occurs secondary to cancer chemotherapy with these agents. Chemotherapy induced adverse effects include cognitive effects (especially reduced cognition or cognition deficit, foggy brain, diminished concentration and short- and long-term memory loss) and adverse effects which are caused by myelin degradation.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the term neoplasia is used to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell hung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain preferred aspects, the cancer which is treated is lung cancer, breast cancer, ovarian cancer and/or prostate cancer. In other embodiments, the cancer is breast, ovarian, prostate, cervical (especially during pregnancy), testicular, head and neck cancer, Hodgkin's lymphoma, non-small cell lung cancer, lymphoma, brain cancer, neuroblastoma, leukemia, solid tumors, cancer of the bladder, stomach, thyroid, soft tissue sarcoma, multiple myeloma colon cancer, esophageal cancer, stomach cancer or pancreatic cancer.

As used herein, the terms malignant neoplasia and cancer are used synonymously to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, stomach and thyroid; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid, cyclophosphamide, Adriamycin or 5-flurouracil or agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, agent(s) which may be co-administered with compounds according to the present invention in the treatment of cancer. These agents include chemotherapeutic agents and include one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, and HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor a Map kinase kinase (mek) inhibitor, a VEGF trap antibody, pemetrexed, erlotinib dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, eilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-IC11, CHIR-258,); 3-[5-(methylsulfonylpiperadinemethyl)-indolylj-quinolone, vatalanib, AG-013736, AVE-0005, the acetate salt of [D-Ser(But)6,Azgly 10] (pyro-Glu-His-Trp-Ser-Tyr-D-Ser(But)-Leu-Arg-Pro-Azgly-NH₂ acetate [C₅₉N₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, parnidronate, pentostatin, plicamycin, porfimer, procarbazine, raltitrexed, rituximab, streptozocin, teniposide, testosterone thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11 dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin, alfa, ipilimumab, nivolomuab, pembrolizumab, dabrafenib, trametinib and vemurafenib among others.

The term “co-administration” or “combination therapy” is used to describe a therapy in which at least two active compounds in effective amounts are used to treat cancer and/or CIAE, especially including central nervous system adverse effects such as chemobrain, impaired cognition, loss of concentration, short- or long-term memory loss, or another disease state or condition as otherwise described herein, either at the same time or within dosing or administration schedules defined further herein or ascertainable by those of ordinary skill in the art. Although the term co-administration preferably includes the administration of two active compounds to the patient at the same time (contemporaneously, concominantly or sequentially), it is not necessary that the compounds be administered to the patient at exactly same time, although effective amounts of the individual compounds will be present in the patient at the same time. In addition, in certain embodiments, co-administration will refer to the fact that two compounds are administered at significantly different times, but the effects of the two compounds are present at the same time. Thus, the term co-administration includes an administration in which one active agent (especially a PKC inhibitor or a PKC inhibitor in combination with lithium) are administered at approximately the same time (contemporaneously), or from about one to several minutes to about eight hours, about 30 minutes to about 6 hours, about an hour to about 4 hours, or even much earlier than the CIAE-inducing anti-cancer active ingredient as otherwise described herein including up to a day or substantially more (3-5 days or more up to ten days or alternatively, several days). It is noted that in certain embodiments, the PKC inhibitor or the PKC inhibitor in combination with lithium may be administered before (pre-administration) or after (post-administration) the CIAE-inducing anti-cancer active ingredient and still have an ameliorative or protective effect. This effect may occur even when the PKC or PKC and lithium salt are administered from one hour to up to 3-5 days before (pre-administration) and from one hour to up to 10 days and often a day to about 10 days after (post-administration) the administration of the CIAE-inducing anti-cancer active ingredient.

Co-administered anticancer compounds can include, for example, Aldesleukin; Alemtuzumab; alitretinoin; allopurinol; altretamine; amifostine; anastrozole; arsenic trioxide, Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate, Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemtuzumab ozogamicin; gleevec (imatinib); goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; surafenib; talbuvidine (LDT); talc; tamoxifen; tarceva (erlotinib); temozolomide; teniposide (VM-26); testolactone; thioguanine (6-TG); thiotepa; topotecan; toremifene; Tositumomab; Trastuzumab; tretinoin (ATRA); Uracil Mustard; valrubicin; valtorcitabine (monoval LDC); vinblastine; vinorelbine; zoledronate; and mixtures thereof, among others.

Co-administration of one of the formulations of the invention with another anticancer agent will often result in a synergistic enhancement of the anticancer activity of the other anticancer agent, an unexpected result. In addition, the co-administration of an effective amount of a PKC inhibitor and a lithium salt produces a favorable therapeutic or prophylactic synergistic effect on chemobrain. One or more of the present formulations may also be co-administered with another bioactive agent (e.g., antiviral agent, antihyperproliferative disease agent, agents which treat chronic inflammatory disease, among others as otherwise described herein).

“Tyrosine kinase inhibitors” may also be used in combination anticancer therapy according to the present invention and include, but are not limited to imatinib, axitinib, bosutinib, cediranib, dasatinib, erlotinib, gefitinib, lapatinib, lestaurtinib, nilotinib, semaxanib, sunitinib, toceranib, vandetanib, vatalanib, sorafenib (Nexavar®), lapatinib, motesanib, vandetanib (Zactima®), MP-412, lestaurtinib, XL647, XL999, tandutinib, PKC412, AEE788, OSI-930, OSI-817, sunitinib maleate (Sutent®)) and N-(4-(4-aminothieno[2,3-d]pyrimidin-5-yl)phenyl)-N′-(2-fluoro-5-(trifluor-omethyl)phenyl)urea, the preparation of which is described in United States Patent Application Document No. 2007/0155758.

Pharmaceutical compositions comprising combinations of an effective amount of at least one anti-cancer active ingredient (e.g., a taxane, vinca alkaloid, cyclophosphamide, Adriamycin/doxorubicin and/or 5-Fluorouracil) and at least one PKC inhibitor and optionally a lithium salt, all in effective amounts in compositions according to the present invention. In addition, one or more other additional anti-cancer compounds as otherwise described herein, all in effective amounts, may be included in pharmaceutical compositions according to the present invention. Each composition may further (preferably) include a pharmaceutically effective amount of a carrier, additive and/or excipient.

The compositions used in methods of treatment of the present invention, and pharmaceutical compositions of the invention, may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions used in methods of treatment of the present invention, and pharmaceutical compositions of the invention, may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions used in methods of treatment of the present invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin cancers. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and the type of cancer treated, and the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of at least one anti-cancer active ingredient and at least one PKC inhibitor and optional lithium salt, further optionally in combination with at least one additional anti-cancer active ingredient. In alternative embodiments, the pharmaceutical composition comprises at least one PKC inhibitor in combination with at least one lithium salt, all in effective amounts to reduce the likelihood, inhibit or reverse cognitive impairment caused by anti-cancer treatment with one or more of a taxane, vinca alkaloid, cyclophosphamide, Adriamycin or 5-Fluorouracil.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

These and other aspects of the invention are described further in the following non-limiting examples.

### EXAMPLES

The number of cancer survivors has increased rapidly due to improvements in awareness, screening, prevention, diagnosis, and treatment (1). Yet, cancer treatments are associated with severe, long-lasting, and sometimes irreversible side effects. Recent evidence from structural (2, 3) and functional (4-6) imaging studies on cancer survivors shows that chemotherapy-induced cognitive impairment, or “chemobrain,” affects between 17% and 75% of cancer survivors (7), some many years after treatment ends. Symptoms of chemobrain include memory lapses, learning difficulties, and troubles with focusing, planning, and multitasking (8-10). With an estimated 16 million cancer survivors in the US alone (11), preventing or alleviating chemobrain is an urgent clinical need. Because the onset of the neurological insult, which is the start of chemotherapy, is known, the initiation phase of chemobrain is a promising timepoint for intervention. Determining the cellular and molecular mechanisms of chemobrain will also facilitate the discovery of better prevention and treatment options.

In these examples, we focus on paclitaxel, which is often the first-line treatment for prevalent cancer types, including breast cancer, ovarian cancer (12-14), and other solid cancers (15, 16). The antitumor effect of paclitaxel is attributed to the stabilization of tubulin polymers (17), causing mitotic arrest and apoptosis (18). However, paclitaxel is responsible for numerous side effects that appear to be tubulin-independent, including peripheral neuropathy (19). We previously elucidated a mechanism for paclitaxel-induced peripheral neuropathy, in which paclitaxel binds neuronal calcium sensor 1 (NCS1) to induce spontaneous InsP3R-dependent calcium oscillations (20-27). Through blocking calcium oscillations (21), lithium pretreatment rescued paclitaxel-induce peripheral neuropathy in a mouse model (26). Lithium is a clinically approved drug for the treatment of depression and bipolar disorders since the 1950s (28), and has been shown to be beneficial in animal models of TBI, aging, Alzheimer's disease, and other neurodegenerative diseases (29). Recent studies found that paclitaxel and its analog docetaxel can penetrate the blood-brain barrier and accumulate in the central nervous system (CNS) (30-32). Furthermore, dysregulated calcium release via the InsP3R has been implicated in cognitive impairment in Alzheimer's disease (AD) (33) and psychological stress (34). Therefore, we aimed to further investigate the effect of paclitaxel in the CNS. We hypothesized that the mechanism and successful treatment with lithium we observed for paclitaxel-induced peripheral neuropathy would also apply to cognitive impairment.

In this study, we established a mouse model of chemobrain in which 4 injections of 20 mg/kg of paclitaxel impaired short-term spatial memory acquisition in mice both acutely at 5 days post-injection (DPI), and chronically at 23 DPI. Using Golgi-Cox staining, we observed altered neuronal morphology in the dentate gyrus and the frontal cortex. We also found an upregulation of protein kinase C α (PKCα), an effector in the InsP3R signaling pathway, acutely in the cortex and hippocampus, and chronically in the cortex. Pretreatment with lithium or the PKC inhibitor chelerythrine rescued deficits induced by paclitaxel injections. Additionally, posttreatment with lithium up to 10 days after paclitaxel injection reversed the memory deficits, but not when administered later, suggesting that there is a limited time window for rescuing chemobrain. Overall, we provide evidence that dysregulation in the InsP3R calcium signaling pathway and disruption of neuronal morphology contribute to paclitaxel-induced cognitive impairment, and that targeting this pathway is a promising approach to prevent chemobrain.

### Materials and Methods

### Animal Use and Treatment

This study was carried out in accordance with the recommendations in the U.S. National Institutes of Health Guide for the Care and Use of Laboratory Animals. The protocol was approved by the Institutional Animal Care and Use Committee at Yale University, and all efforts were made to minimize suffering. Mice were maintained on a 12:12-h light/dark cycle (7:00 am on/7:00 pm off) with food and water provided ad libitum before experimental procedures. All animal experiments were performed during the light cycle.

### Paclitaxel-Induced Model of Cognitive Impairment

Seven-week-old female C57BL/6 mice were purchased from Charles River (Wilmington, Mass., USA) and allowed to habituate to the facility for 7 days, followed by 3 days of handling before the start of the experiment. Mice were randomly assigned into groups, with all groups represented in each cage. Depending on the treatment group, lithium chloride (Sigma-Aldrich, 12.8 mg/kg in 0.9% saline), chelerythrine chloride (Cayman Chemical, 2 mg/kg in 0.4% DMSO in saline), or the appropriate vehicle was administered intraperitoneally 1 h before injection of vehicle (20% 50:50 Cremophor EL: ethanol, 80% saline) or paclitaxel (Cayman Chemical, 20 mg/kg in 20% 50:50 Cremophor EL: ethanol, 80% saline) to induce cognitive impairment. Each mouse received a total of 4 pairs of injections over 8 days. During and after injections, mice were weighed daily and checked for general health and any sign of pain or distress.

### Open-Field Exploration and Displaced Object Recognition

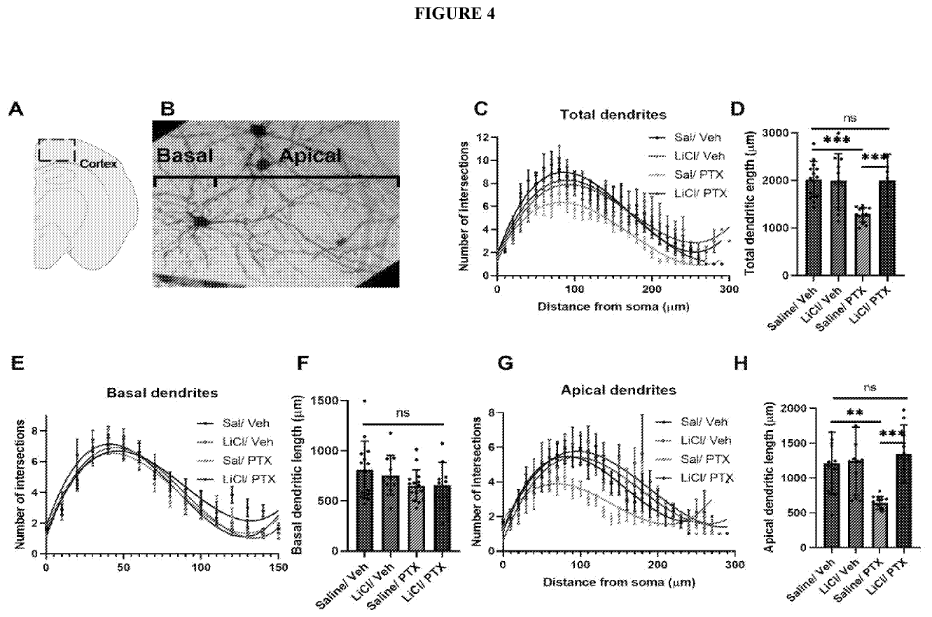

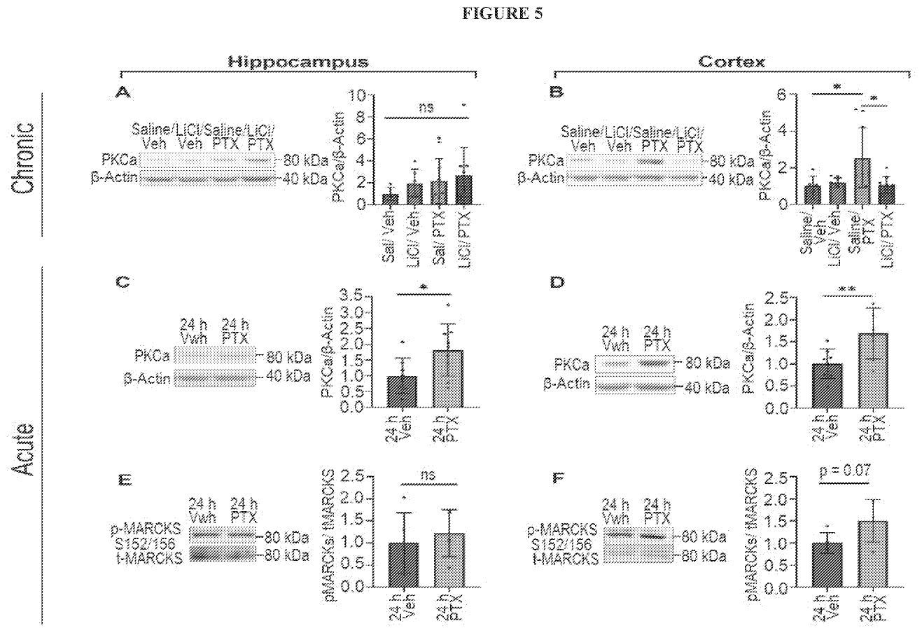

Behavioral experiments were adapted from a published protocol (35). Data were analyzed blinded to the experimental conditions. Open-field exploration (OFE) and displaced object recognition (DOR) tasks were carried out over two consecutive days (FIG. 4.1). The experimental arena was a 35×70×35 cm opaque, white Plexiglas chamber. The arena was covered with ˜1 cm of standard corn cob bedding. After each mouse, feces were removed, and the bedding was shaken to distribute odor cues equally. A camera was mounted 100 cm above the arena to record the test sessions. The test was conducted during the mice's light phase under low light condition (45 Lux). 1 h before testing, mice were brought up and allowed to habituate to the testing room.

For the OFE task, each mouse was allowed to explore the arena for 10 min. The camera footage was then analyzed using ToxTrac, a published program for the total distance moved and time spent in the peripheral versus the central areas (36). For the DOR task, pairs of 50-mL tubes filled with corn cob bedding were taped cap-down to pre-determined positions in the arena. They were selected specifically because mice were unable to climb onto the pointed ends of the tubes. During the familiarization phase, each mouse was first allowed to explore the arena where the two Falcon tubes were placed in symmetrical locations for 5 min before being taken out and returned to its home cage. 2 h later, the mouse was returned to the arena for another 5 min, with 1 tube remaining in the same position and 1 tube moved to a different position. The positions of the tubes were counterbalanced. After each mouse, the tubes were sprayed with water and wiped dry to remove odor cues. The camera footage was then analyzed for bouts of interactions with the tubes. Sniffing and biting were considered to be interaction. Casual touching of the tubes in passing, or leaning onto the tubes to look around were not counted. The preference index for the displaced object was calculated as 100*(time spent with displaced object)/(total time spent with both displaced and familiar objects). The preference index for the familiar object was similarly calculated.

### Euthanasia and Tissue Collection

Golgi-Cox staining solutions were prepared in advance, according to a published protocol (37). For tissue collection, each mouse was first anesthetized for ˜30 s with 30% isoflurane, and then quickly decapitated with scissors. The skull was opened, and the brain was extracted and washed briefly in ice-cold 1× phosphate-buffered saline solution (PBS, AmericanBio). A razor blade was then used to dissect the brain into two hemispheres along the medial longitudinal fissure. One hemisphere was immediately dropped into a 25-mL scintillation vial containing 10 mL of impregnation stock solution. The other hemisphere was rapidly dissected into the cerebellum, hippocampus, frontal cortex, and midbrain, snap-frozen in liquid nitrogen, and then subsequently stored at −80° C. until further use.

### Golgi-Cox Staining, Imaging, and Quantification

Golgi-Cox staining was performed according to a published protocol (37). Briefly, the samples were impregnated with a potassium dichromate and mercuric chloride solution at room temperature for 7 days, followed by immersion in a cryoprotection solution for 4 days, and then sectioned into 200 μM frontal slices with a vibratome. The Golgi Atlas of the Postnatal Mouse Brain was used as the reference to identify the section position of the slices (38). Slices corresponding to frontal sections 10 and 11 in the atlas were selected for imaging the hippocampus and the parietal cortex. Slices corresponding to frontal sections 4 and 5 were selected for imaging the prefrontal cortex. The selected slices were mounted on 0.3% gelatin-coated slides, developed and dehydrated through a series of increasing alcohol concentrations, then with xylene, and finally mounted in Eukitt solution (Sigma Aldrich) for imaging. Z-stack images of different regions, including the dentate gyrus and the frontal cortex, were collected using a Zeiss LSM 710 Duo microscope with 20× and 60× objectives. Neurons that showed intact and complete dendritic arbors, consistent dark staining, and relative isolation other neurons, were selected for imaging. Spines were imaged from basal dendritic branches at least 50 μM away, and apical dendritic branches at least 100 μM away from the cell soma. Scholl analysis, total dendritic length, number of branch points, and spine density were performed with ImageJ using the Simple Neurite Tracer plugin (39). Spine density was quantified as the number of protrusions on dendritic branches per μm dendrite length

### Tissue Lysis and Western Blot

Frozen tissues were thawed in RIPA buffer containing protease inhibitor, phenylmethylsulfonyl fluoride (PMSF), and sodium orthovanadate (Santa Cruz), homogenized with a polytron, and then spun down twice at 13000 rpm, 4° C. to remove cell debris. Protein concentration was quantified using Pierce BCA protein assay kit (ThermoFisher Scientific) according to the manufacturer's instruction. Western blots were performed using the NuPAGE system (ThermoFisher Scientific) and PVDF membrane with the Biorad wet transfer system (Bio-Rad Laboratories). Approximately 20 μg total protein was loaded into each lane. Information about the antibodies used is included in Supp. Table 1.

### Statistical Analyses

Data management and calculations were performed using PRISM Statistical Software 8 (GraphPad Software, Inc, California). The specific statistic tests were detailed in the figure legend and Supp. Table 2. Generally, two-tailed student t-test was used to compare two groups, one-way ANOVA followed by Tukey's post-hoc test was used to compare multiple groups, and two-way repeated ANOVA followed by Dennet's post-hoc test was used for Sholl analyses. A p-value<0.05 was considered to be statistically significant and the following notations were used in all figures: * for p<0.05, ** for p<0.01, *** for p<0.005, and **** for p<0.0001. For Sholl analysis graphs, error bars shown were standard error of the mean (SEM). For all other graphs, error bars shown were standard deviation.

### Results

### Establishing a Mouse Model of Chemobrain

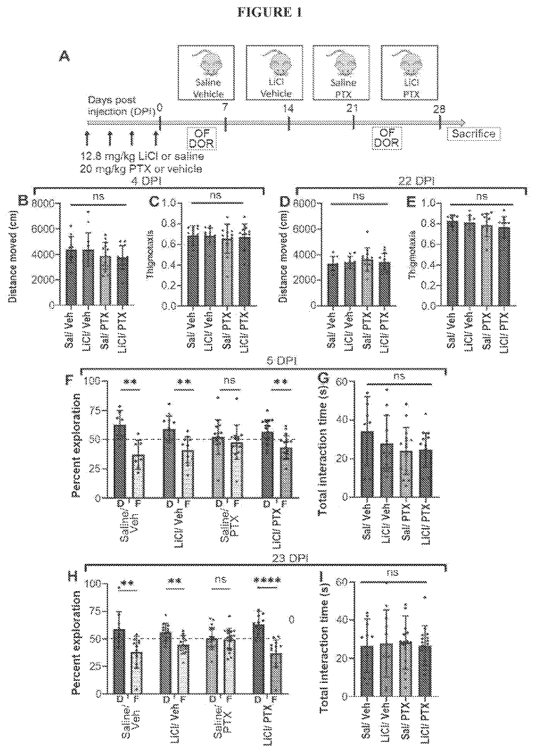

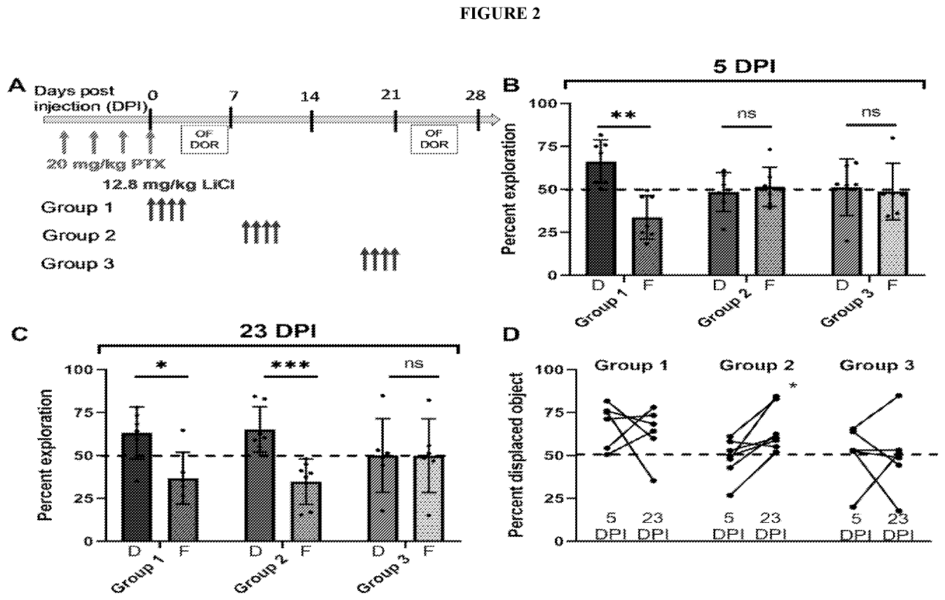

To measure cognitive function, we selected the displaced object recognition task (DOR) task with a 2-h interval between the familiarization and the test sessions. Optimal performance on this task requires contribution from both the hippocampus and the cortex, as either prefrontal cortical or hippocampal lesions were sufficient to impair task performance (40). In addition, the short inter-session interval also puts greater emphasis on cortex-dependent working and short-term memory instead of long-term memory consolidation in the hippocampus (41). After optimization, we found that 4×20 mg/kg paclitaxel was sufficient to impair DOR task performance without additional side effects (FIG. 1 & Supp. FIG. 1). No significant differences in weight loss were observed among the groups (Supp. FIG. 1B). 20 mg/kg paclitaxel translates to ˜60 mg/m² per dose in humans, which is below the recommended maximum 175 mg/m² dose per visit (42). We optimized the dose of lithium in our model and found that pretreatment with 12.8 mg/kg LiCl rescued the performance in the DOR task, both 5 DPI and 23 DPI (Supp. FIG. 1C). This dose resulted in peak plasma lithium level of 0.36 mM (Supp. FIG. 2), which is below the lower therapeutic target range (0.5 to 0.8 mM) in humans (43).

### Lithium Pretreatment Rescues Paclitaxel-Induced Short-Term Memory Impairment

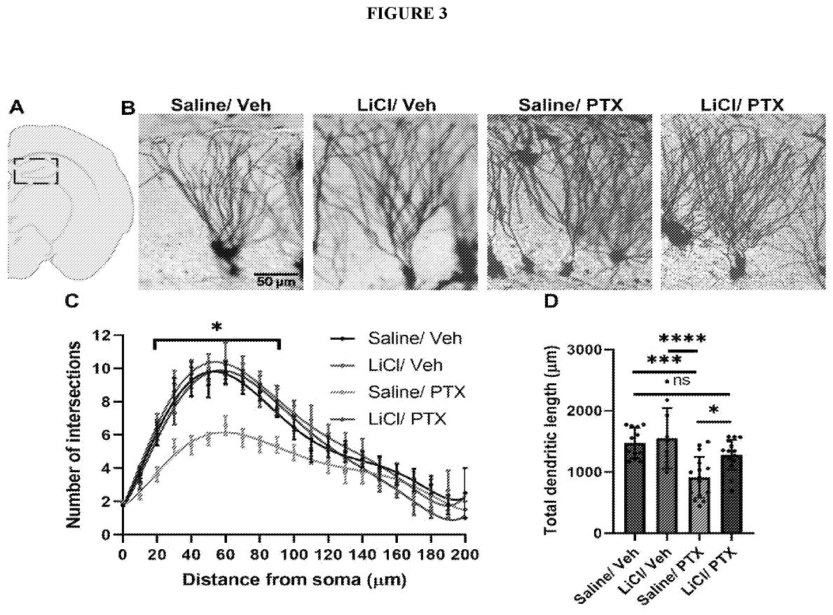

Mice were randomly divided into 4 groups, with each group receiving either saline or 12.8 mg/kg lithium, and then 1 h later, either vehicle or 20 mg/kg paclitaxel. Mice received a total of 4 pairs of injections, 1 pair every two days. No significant weight loss was observed in all groups over the injection duration (Supp. FIG. 3). Mice were tested with the open-field exploration (OFE) and the DOR tasks at 4 and 5 DPI respectively to measure the acute effects of paclitaxel toxicity. Both tests were then repeated at 22 and 23 DPI to measure the chronic effects (FIG. 1A). The OFE task measured locomotor performance through the total distance traveled, as well as anxiety through the thigmotaxis index. A higher thigmotaxis index indicates higher levels of anxiety (44). We observed no differences among the groups (FIG. 1, B-E), suggesting that the paclitaxel dose used neither impaired locomotor activity nor caused increased anxiety.

Short-term memory impairment was measured using the DOR task. At 5 DPI (FIG. 1F), control mice receiving only vehicle injection with saline or lithium showed a significant preference for the displaced object (two-tailed student t-test, corrected for multiple comparisons, p=0.004 and p=0.002 respectively). In contrast, mice receiving paclitaxel with saline showed no preference for either object (p=0.4), suggesting that they had impaired short-terns memory acquisition. Similar to what we previously reported for the peripheral nervous system (26), lithium pretreatment also rescued the preference for the displaced object (p=0.003). Total interaction time was similar among all groups, suggesting that this was not a confounding factor (FIG. 1G). The memory deficits in mice receiving paclitaxel persisted up to 23 DPI (FIGS. 1, H and I), suggesting long-lasting impairment, which could be prevented by lithium pretreatment.

### Within a Limited Time Window, Lithium Treatment After Paclitaxel Reverses Memory Impairment

To investigate whether lithium can also alleviate chemobrain when administered after patients finished chemotherapy and, if so, what the therapeutic window would be, we investigated several posttreatment schedules in our model. Mice were divided into 3 groups, with all groups receiving 4×20 mg/kg paclitaxel. Subsequently, groups received 4 doses of 12.8 mg/kg LiCl at 0-3 DPI, 7-10 DPI, or 17-20 DPI, respectively, and assessed with the established schedule of DOR (FIG. 2A). Group 1, which received lithium immediately after paclitaxel, showed normal memory acquisition both at 5 DPI and 23 DPI (FIGS. 2, B and C). Importantly, group 2, which showed impaired memory acquisition at 5 DPI (FIG. 2B), subsequently showed normal memory acquisition when tested at 23 DPI (FIG. 2C). When examined individually, the majority of mice in group 2 developed a significantly greater preference for the displaced object after the lithium treatment (FIG. 2D, paired two-tailed student t-test, p=0.03). However, group 3, which showed impaired memory acquisition at 5 DPI and received lithium more than two weeks after injection, showed no improvement at 23 DPI (FIG. 2D, p=0.9), suggesting that lithium given later was insufficient to reverse cognitive impairment. These data indicate that, within a limited time window, lithium can not only prevent but also reverse chemobrain.

### Paclitaxel Reduces Hippocampal Neuron Complexity

The inventors also investigated possible neural correlates underlying chemobrain. Various chemotherapeutics, including cisplatin (45), fluorouracil (46), doxorubicin, and cyclophosphamide (47, 48), were reported to reduce the dendritic complexity in granule cells, and CA1 and CA3 pyramidal neurons in the hippocampus. Therefore, we performed Golgi-Cox staining of mouse brain hemispheres, and subsequently, Sholl analysis on dentate gyrus granule cells to examine changes in dendritic complexity as a function of the number of intersections at various radial distances from the cell soma (FIG. 3, A-C). Two-way repeated-measures ANOVA revealed a significant effect of treatment (F (3, 59)=5.970, p=0.0013), distance from the soma (F (4.370, 198.2)=54.46, p<0.0001), as well as significant interaction between treatment and distance (F (60, 907)=1.624, p=0.0024). Post-hoc analysis revealed that saline/paclitaxel neurons showed reduced dendritic arborization compared to saline/vehicle controls, particularly between 20-90 μm (Dunnett's multiple comparisons test, p<0.05 at each position). In addition, there was also a significant reduction in the total dendritic length in the saline/paclitaxel group comparing to the other groups (FIG. 3D, one-way ANOVA, followed by Tukey post-hoc test). No significant differences were found comparing saline/vehicle neurons with lithium/vehicle or lithium/paclitaxel neurons. These results suggest that paclitaxel injections reduced hippocampal dendritic complexity, which was rescued with lithium pretreatment.

### Paclitaxel Reduces Apical Cortical Neuron Complexity

As worse performance in our DOR task could suggest impairments in both the frontal cortex and the hippocampus, we also performed Golgi-Cox staining and Sholl analysis on layers 2/3 cortical pyramidal neurons in the medial prefrontal cortex (FIGS. 4, A and B). Because cortical pyramidal neurons exhibit both basal and apical dendrites, each with distinct functions and input sources (49, 50), we performed analyses separately for each region. For basal dendrites, we observed no significant differences in the Sholl analysis (FIG. 4C, two-way repeated-measures ANOVA, for treatment, F (3, 92)=0.33, p=0.81) and dendritic length among the 4 groups (FIG. 4D, one-way ANOVA, p=0.23). Similarly, there were no significant differences in basal spine density (FIGS. 4, E and F, one-way ANOVA, p=0.94).

In contrast, for apical dendrites, significant differences were observed in the Sholl analysis (FIG. 4G, for treatment, F (4.857, 358.4)=29.40, p<0.0001; for distance, F (3, 92)=9.159, p<0.0001). The interaction between treatment and distance from the cell soma was also significant (F (87, 2140)=1.537, p=0.01). Post-hoc analysis revealed that saline/paclitaxel neurons showed reduced dendritic arborization compared to saline/vehicle controls, particularly between 110-230 μm (Dunnett's multiple comparisons test, p<0.05 at each position). Similarly, the saline/paclitaxel group exhibited a significant reduction in apical dendritic length and spine density compared to the other three groups (FIG. 4, H-J). These results suggest that apical dendrites were specifically susceptible to paclitaxel, whereas basal dendrites were largely spared. Similar results were also observed for neurons imaged in the parietal cortex (Supp. FIG. 4), suggesting that other cortical areas are also affected.

### Paclitaxel Upregulates PKCα

Next, we focused on molecular changes that may underly chemobrain. Particularly, we investigated changes in the InsP3R pathway, which we hypothesized to be dysregulated by paclitaxel. We observed an upregulation in PKCα, an effector of the InsP3R pathway, in the cortex of mice treated with paclitaxel at 30 DPI (FIG. 5B, one-way ANOVA, saline/veh vs. saline/paclitaxel=0.026), but not in the hippocampus (FIG. 5A, p=0.51). No changes were observed for other proteins involved in the InsP3R pathway, including InsP3R1, NCS1, phospholipase C (PLC-β1) (Supp. FIG. 5).

To assess molecular changes involved in the initiation of chemobrain, we also collected tissues from mice 24 h after a single injection of 20 mg/kg paclitaxel injection or vehicle control. An upregulation in PKCα was again observed in both the hippocampus (FIG. 5C, two-tailed student t-test, p=0.03) and the cortex (FIG. 5D, p=0.009). To measure possible downstream functional consequences of PKCα activity, we examined the phosphorylated form of the PKC substrate myristoylated alanine-rich C-kinase substrate (MARCKS) (51). There was a trend towards increased pMARCKS (S152/156) in the cortex 24 h after paclitaxel injection (FIG. 5F, p=0.07), but not in the hippocampus (FIG. 5E, p=0.6). Taken together, our molecular analyses suggest that PKCα contributes to the behavioral and cellular deficits in mice receiving paclitaxel.

Pretreatment with PKC Inhibitor Chelerythrine Rescues Paclitaxel-Induced Short-Term Memory Impairment

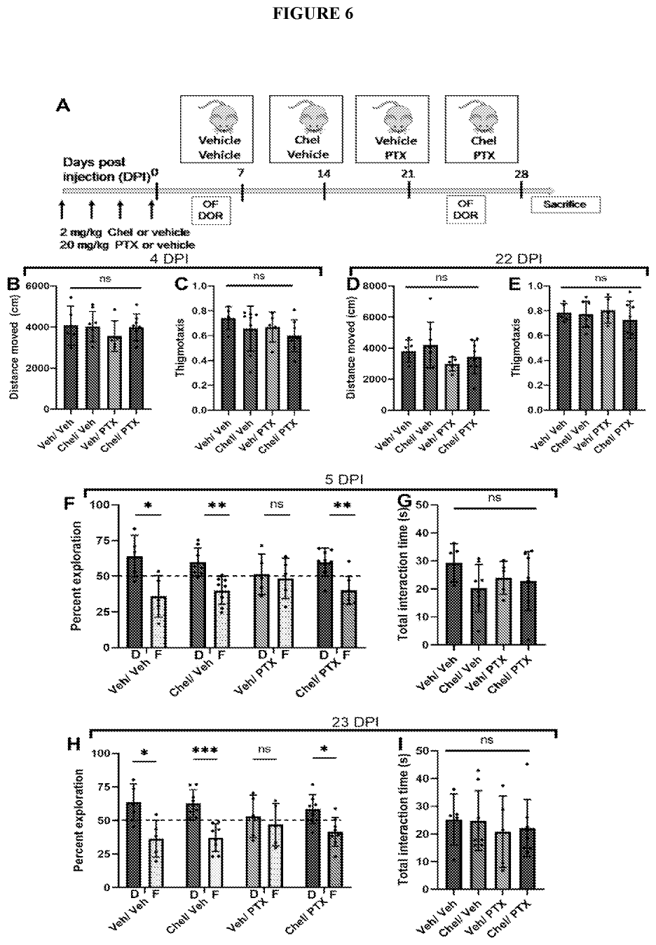

It was previously shown that chronic restraint stress in rats resulted in calcium-dependent activation of PKC activity, leading to reduced cortical spines and dendrites, and hence impaired working memory (52). Furthermore, pretreatment with a brain-penetrant PKC inhibitor, chelerythrine, rescued the working memory. Therefore, to test the hypothesis that upregulation of PKC expression, as well as calcium-induced activation of PKC activity, contributes to paclitaxel-induced memory impairment, we examined whether pretreatment with chelerythrine could prevent memory deficits in our model of chemobrain. Pretreatment with chelerythrine resulted in similar results to pretreatment with lithium (FIG. 6). First, no differences in total distance moved and thigmotaxis were observed among the four groups at both 4 DPI and 22 DPI (FIG. 6A-E). Second, pretreatment with chelerythrine prevented paclitaxel-induced memory impairment at both 5 DPI and 23 DPI, whereas chelerythrine alone did not affect memory acquisition (FIG. 6F-I). These results further underscore that chelerythrine and lithium act in a similar pathway to rescue paclitaxel-induced short-term memory impairment.

### Discussions

### Lithium for the Prevention and Treatment of Paclitaxel-Induced Cognitive Impairment

As presented, we successfully established that treatment with lithium both before and after paclitaxel injections rescued cognitive impairment. Our results agree with previous studies reporting that lithium pretreatment rescues paclitaxel-induced peripheral neuropathy (26, 53) and cognitive impairment (32). To the best of our knowledge, we are the first to report that posttreatment with lithium, albeit within a limited time window, can reverse the cognitive deficits induced by paclitaxel. We speculate that the window of effectiveness of lithium treatment matches the trajectories of the mechanisms underlying paclitaxel-induced cognitive deficits. Similar to traumatic brain injury, chemobrain is initiated by an acute insult, which is the administration of paclitaxel (54). This initiating phase is then followed by the chronic phase, in which deficits are consolidated and maintained even when the original source of insult is gone. The mechanisms of action of lithium remain varied and incompletely understood. Although we previously showed that lithium blocks paclitaxel-induced InsP3R calcium oscillation (21), lithium also inhibits inositol monophosphatase (29) and PKC (55, 56) to further downregulate the InsP3R calcium pathway. Lithium appears to interfere with the initiation and consolidation of chemobrain, and become less effective further over time as the cellular and molecular deficits become permanent. An alternative explanation is that in our experimental design (FIG. 2A), group 3 received lithium only 3 days before the chronic DOR task. However, more time is needed between lithium treatment and DOR task before improvements can be observed. Further experiments will be needed to clarify this question.

### A Role for PKC Hyperactivation in Cellular and Behavioral Deficits

We observed a reduction in dendrite complexity and length in the hippocampus and cortex of mice treated with paclitaxel, which we hypothesize to be the cellular mechanism for the memory deficits observed in these mice. The upregulation of PKCα acutely in the hippocampus and the cortex, which persists chronically in the cortex, may provide the underlying molecular mechanism for this observation. PKCα activity can also be activated by elevation in calcium (57). PKCα hyperactivity has been implicated in stress and age-induced loss of dendritic and spinal complexity and cognitive deficits (52, 57, 58). Furthermore, PKC isoforms have been shown to play a role in paclitaxel-induced peripheral neuropathy (59). We also observed the trend towards the upregulation of PKC substrate, pMARCKS. Phosphorylation of MARCKS has been shown to cause dendrite and spine loss through inducing actin instability (60). Interestingly, paclitaxel, but not other chemotherapeutic drugs, was shown to dose-dependently increase pMARCKS in breast cancer cell lines (61). As a proof of concept, we showed that pretreatment with the PKC inhibitor chelerythrine rescued memory deficits without affecting the behaviors of animals not receiving paclitaxel.

### Region-Specific Vulnerability Chemotherapy

We previously proposed that determining the specific cognitive modalities, anatomical regions, and cell populations that are more vulnerable to chemotherapy will be essential for discovering prevention and treatment options (54). Interestingly, although paclitaxel was reported to preferentially accumulate in the hippocampus as compared to the cortex (32), we found that neuronal morphology was also altered in the cortex. In the prefrontal cortex, apical dendrites and spine density were reduced, whereas basal dendrites and spines were spared. Persistent activity in layer 2/3 apical dendrites was proposed to be essential for recurrent neural activity, which in turn sustains working memory and attention (62, 63)—cognitive functions that are often impaired in chemobrain. A similar phenomenon of apical vulnerability has been frequently reported in animal models of stress-induced cognitive impairment (52, 64-67), as well as AD (68) and aging (69). Apical dendrites receive input from diverse sources such as higher cortical regions and the thalamus, and function to modulate selectivity (50). In contrast, basal dendrites receive input from more restricted sources such as local pyramidal cells and interneurons, and function to drive stimulus preference (50). Although the cause of selective apical vulnerability remains to be clarified, candidates include differential distribution of molecular machineries, for example, availability of channels and receptors, and altered input into basal or apical dendrites. Our findings suggest that loss of apical spines and dendrites is a neural correlate for chemobrain, and may share similar pathways with cognitive deficits in aging, AD, and psychological stress.

### Proposed Model

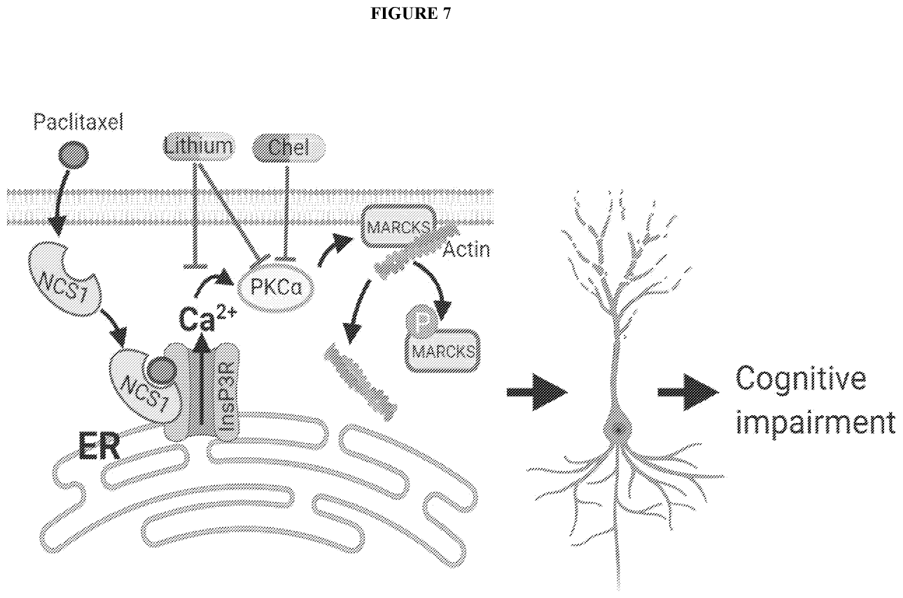

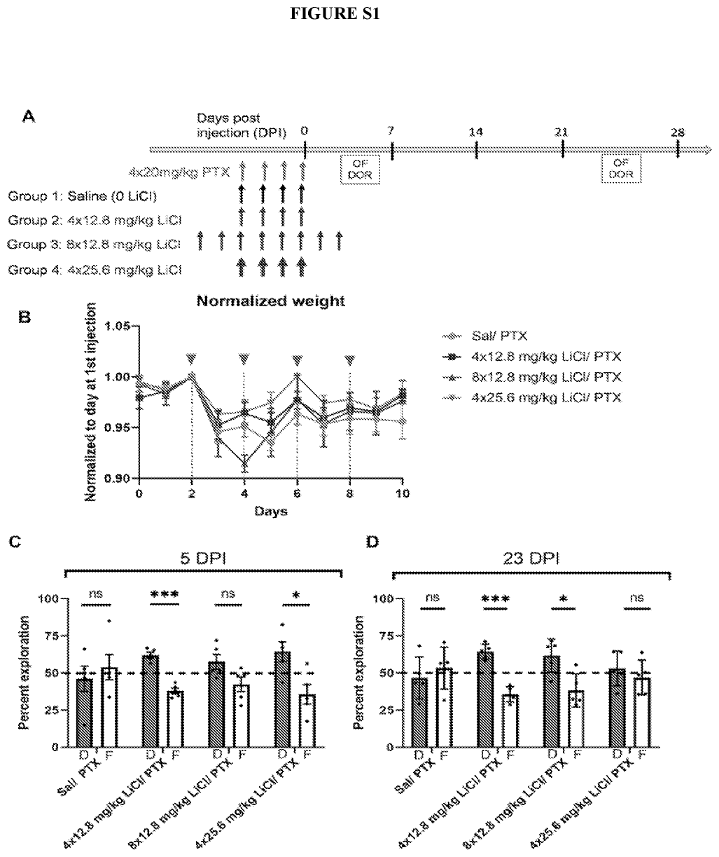

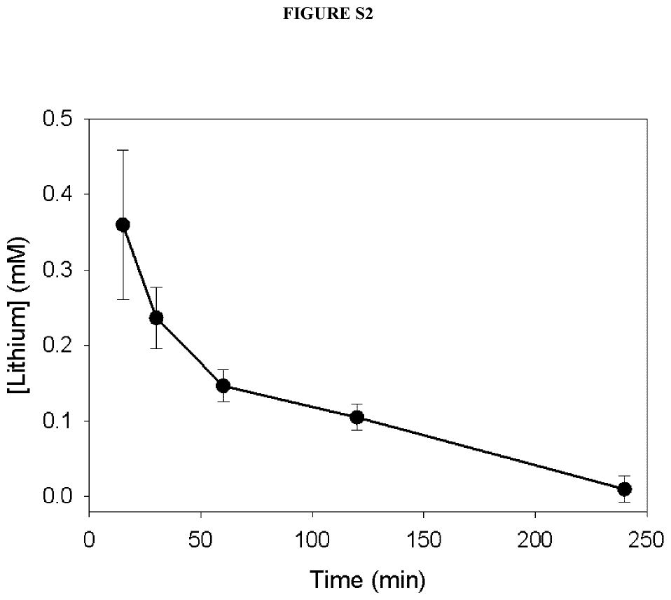

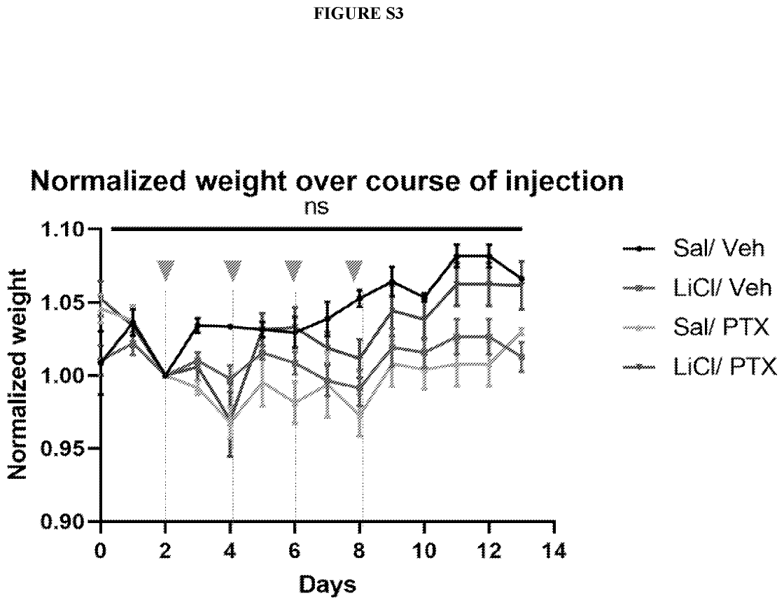

Combining behavioral, cellular, and molecular observations, we propose a model for paclitaxel-induced cognitive impairment and describe how lithium and chelerythrine can interfere with this pathway (FIG. 7). First, paclitaxel binding to NCS1 leads to increased calcium oscillation from the InsP3R (20, 21, 24, 27). This results in the activation of PKC, which phosphorylates MARCKS into pMARCKS, leading to actin instability, and hence spine and dendrite retraction. Lithium interferes with this pathway through either depleting InsP3 to decrease InsP3R activity, or through indirectly blocking PKC activity. Chelerythrine (and other PKC inhibitors) blocks PKC activation and hence blocks MARCKS phosphorylation. Interfering in this pathological pathway rescued dendrite and spine retraction, and consequently prevented memory impairment. Collectively, our findings suggest a pathway for paclitaxel-induced cognitive impairment. Paclitaxel, and potentially other chemotherapeutic drugs, may accelerate neurodegeneration through InsP3R-dependent calcium release, a common pathway for cognitive impairment in aging, psychological stress, and Alzheimer's disease (33, 34). Although the mechanisms of lithium-based therapy remain unclear, its pharmacokinetics are well-studied, and low lithium may be beneficial for chemobrain as it has been shown to be generally neuroprotective (29). Furthermore, the effective lithium dose we used is below the common therapeutic range for treating mood disorders. Taken together, lithium and PKC inhibitors may be good preventions and treatments for chemobrain in cancer survivors.

### Abbreviations

AD: Alzheimer's disease
CNS: central nervous system
DOR: displaced object recognition
DPI: days post injection
InsP3R: inositol 1,4,5-trisphosphate receptor
MARCKS: myristoylated alanine-rich C-kinase substrate
NCS1: neuronal calcium sensor 1
OFE: open-field exploration
PKC: protein kinase C
PTX: paclitaxel

### REFERENCES

1. Noone A M H N, Krapcho M, Miller D, Brest A, Yu M, Ruhl J, Tatalovich Z, Mariotto A, Lewis D R, Chen H S, Feuer E J, Cronin K A (eds) National Cancer Institute. Bethesda, Md., https://seer_cancer.gov/csr/1975_2015/, based on November 2017 SEER data submission, posted to the SEER web site, April 2018. 2018.
2. McDonald B C, Conroy S K, Ahles T A, West J D, Saykin A J. Gray matter reduction associated with systemic chemotherapy for breast cancer: a prospective MRI study. Breast Cancer Res Treat. 2010; 123(3):819-28.
3. McDonald B C, Conroy S K, Smith D J, West J D, Saykin A J. Frontal gray matter reduction after breast cancer chemotherapy and association with executive symptoms: a replication and extension study. Brain Behav Immun. 2013; 30 Suppl:S117-25.
4. de Ruiter M B, Reneman L, Boogerd W, Veltman D J, van Dam F S, Nederveen A J, et al. Cerebral hyporesponsiveness and cognitive impairment 10 years after chemotherapy for breast cancer. Hum Brain Mapp. 2011; 32(8):1206-19.
5. Kesler S R, Kent J S, O'Hara. R. Prefrontal cortex and executive function impairments in primary breast cancer. Arch Neurol. 2011; 68(11):1447-53.
6. Lopez Zunini R A, Scherling C, Wallis N, Collins B, MacKenzie J, Bielajew C, et al. Differences in verbal memory retrieval in breast cancer chemotherapy patients compared to healthy controls: a prospective fMRI study. Brain Imaging Behav. 2013; 7(4):460-77.
7. Wefel J S, Schagen S B. Chemotherapy-related cognitive dysfunction. Curr Neurol Neurosci Rep. 2012; 12(3):267-75.
8. Stewart A, Bielajew C, Collins B, Parkinson M, Tomiak E. A meta-analysis of the neuropsychological effects of adjuvant chemotherapy treatment in women treated for breast cancer. Clin Neuropsychol. 2006; 20(1):76-89.
9. Jim H S, Phillips K M, Chait S, Paul L A, Popa M A, Lee Y H, et al. Meta-analysis of cognitive functioning in breast cancer survivors previously treated with standard-dose chemotherapy. J Clin Oncol. 2012; 30(29):3578-87.
10. Lindner O C, Phillips B, McCabe M G, Mayes A, Wearden A, Varese F, et al. A meta-analysis of cognitive impairment following adult cancer chemotherapy. Neuropsychology. 2014; 28(5):726-40.
11. Miller K D, Nogueira L, Mariotto A B, Rowland J H, Yabroff K R, Alfano C M, et al. Cancer treatment and survivorship statistics, 2019. CA Cancer J Clin. 2019.
12. Spencer C M, Faulds D. Paclitaxel. A review of its pharmacodynamic and pharmacokinetic properties and therapeutic potential in the treatment of cancer. Drugs. 1994; 48(5):794-847.
13. Preston N J. Paclitaxel (Taxol)—a guide to administration. Eur J Cancer Care (Engl). 1996; 5(3):147-52.
14. Michaud L B, Valero V, Hortobagyi G. Risks and benefits of taxanes in breast and ovarian cancer. Drug Saf. 2000; 23(5):401-28.
15. Finley R S, Rowinsky E K. Patient care issues: the management of paclitaxel-related toxicities. Ann Pharmacother. 1994; 28(5 Suppl):S27-30.
16. Mekhail T M, Markman M. Paclitaxel in cancer therapy. Expert Opin Pharmacother. 2002; 3(6):755-66.
17. Schiff P B, Fant J, Horwitz S B. Promotion of microtubule assembly in vitro by taxol. Nature. 1979; 277(5698):665-7.
18. Manfredi J J, Horwitz S B. Taxol: an antimitotic agent with a new mechanism of action. Pharmacol Ther. 1984; 25(1):83-125.
19. Gornstein E, Schwarz T L. The paradox of paclitaxel neurotoxicity: Mechanisms and unanswered questions. Neuropharmacology. 2014; 76 Pt A:175-83.
20. Boehmerle W, Splittgerber U, Lazarus M B, McKenzie K M, Johnston D G, Austin D J, et al. Paclitaxel induces calcium oscillations via an inositol 1,4,5-trisphosphate receptor and neuronal calcium sensor 1-dependent mechanism. Proc Natl Acad Sci USA. 2006; 103(48):18356-61.
21. Schlecker C, Boehmerle W, Jeromin A, DeGray B, Varshney A, Sharma Y, et al. Neuronal calcium sensor-1 enhancement of InsP3 receptor activity is inhibited by therapeutic levels of lithium. J Clin Invest. 2006; 116(6):1668-74.
22. Boehmerle W, Zhang K, Sivula M, Heidrich F M, Lee Y, Jordt S E, et al. Chronic exposure to paclitaxel diminishes phosphoinositide signaling by calpain-mediated neuronal calcium sensor-1 degradation. Proc Natl Acad Sci USA. 2007; 104(26):11103-8.
23. Blachford C, Celic A, Petri E T, Ehrlich B E. Discrete proteolysis of neuronal calcium sensor-1 (NCS-1) by mu-calpain disrupts calcium binding. Cell Calcium. 2009; 46(4):257-62.
24. Zhang K, Heidrich F M, DeGray B, Boehmerle W, Ehrlich B E. Paclitaxel accelerates spontaneous calcium oscillations in cardiomyocytes by interacting with NCS-1 and the InsP3R. J Mol Cell Cardiol. 2010; 49(5):829-35.
25. Benbow J H, DeGray B, Ehrlich B E. Protection of neuronal calcium sensor 1 protein in cells treated with paclitaxel. J Biol Chem. 2011; 286(40):34575-82.
26. Mo M, Erdelyi I, Szigeti-Buck K, Benbow J H, Ehrlich B E. Prevention of paclitaxel-induced peripheral neuropathy by lithium pretreatment. FASEB J. 2012; 26(11):4696-709.
27. Nguyen L D, Petri E T, Huynh L K, Ehrlich B E. Characterization of NCS1-InsP3R1 interaction and its functional significance. J Biol Chem. 2019; 294(49):18923-33.
28. Shorter E. The history of lithium therapy. Bipolar Disord. 2009; 11 Suppl 2:4-9.
29. Forlenza O V, De-Paula V J, Diniz B S. Neuroprotective effects of lithium: implications for the treatment of Alzheimer's disease and related neurodegenerative disorders. ACS Chem Neurosci. 2014; 5(6):443-50.
30. Fardell J E, Zhang J, De Souza R, Vardy J, Johnston I, Allen C, et al. The impact of sustained and intermittent docetaxel chemotherapy regimens on cognition and neural morphology in healthy mice. Psychopharmacology (Berl). 2014; 231(5):841-52.
31. Atarod D, Eskandari-Sedighi G, Pazhoohi F, Karimian S M, Khajeloo M, Riazi G H. Microtubule Dynamicity Is More Important than Stability in Memory Formation: an In Vivo Study. J Mol Neurosci. 2015; 56(2):313-9.
32. Huehnchen P, Boehmerle W, Springer A, Freyer D, Endres M. A novel preventive therapy for paclitaxel-induced cognitive deficits: preclinical evidence from C57BL/6 mice. Transl Psychiatry. 2017; 7(8):e1185.
33. Berridge M J. The Inositol Trisphosphate/Calcium Signaling Pathway in Health and Disease. Physiol Rev. 2016; 96(4):1261-96.
34. Amsten A F. Stress weakens prefrontal networks: molecular insults to higher cognition. Nat Neurosci. 2015; 18(10):1376-85.
35. Vogel-Ciernia A, Wood M A. Examining object location and object recognition memory in mice. Curr Protoc Neurosci. 2014; 69:8 31 1-17.
36. Rodriguez A, Zhang H Q, Klaminder J, Brodin T, Andersson P L, Andersson M. ToxTrac: A fast and robust software for tracking organisms. Methods Ecol Evol. 2018; 9(3):460-4.
37. Zaqout S. Kaindl A M. Golgi-Cox Staining Step by Step. Front Neuroanat. 2016; 10:38.
38. Valverde F. Golgi atlas of the postnatal mouse brain. Wien; New York: Springer-Verlag; 1998. xii, 146 p. (some folded) p.
39. Longair M H, Baker D A, Armstrong J D. Simple Neurite Tracer: open source software for reconstruction, visualization and analysis of neuronal processes. Bioinformatics. 2011; 27(17):2453-4.
40. DeVito L M, Eichenhaum H. Distinct contributions of the hippocampus and medial prefrontal cortex to the “what-where-when” components of episodic-like memory in mice. Behav Brain Res. 2010; 215(2):318-25.
41. Antunes M, Biala. G. The novel object recognition memory: neurobiology, test procedure, and its modifications. Cogn Process. 2012; 13(2):93-110.
42. Nair A B, Jacob S. A simple practice guide for dose conversion between animals and human. J Basic Clin Pharm. 2016; 7(2):27-31.
43. Sproule B. Lithium in bipolar disorder: can drug concentrations predict therapeutic effect? Clin Pharmacokinet. 2002; 41(9):639-60.
44. Seibenhener M L, Wooten M C. Use of the Open Field Maze to measure locomotor and anxiety-like behavior in mice. J Vis Exp. 2015(96):e52434.
45. Andres A L, Gong X, Di K, Bota D A. Low-doses of cisplatin injure hippocampal synapses: a mechanism for ‘chemo’ brain? Exp Neural. 2014; 255:137-44.
46. Groves T R, Farris R, Anderson J E, Alexander T C, Kiffer F, Carter G, et al. 5-Fluorouracil chemotherapy upregulates cytokines and alters hippocampal dendritic complexity in aged mice. Behav Brain Res. 2017; 316:215-24.
47. Kang S, Lee S, Kim J, Kim J C, Kim S H, Son Y, et al. Chronic Treatment with Combined Chemotherapeutic Agents Affects Hippocampal Micromorphometry and Function in Mice, Independently of Neuroinflammation. Exp Neurobiol. 2018; 27(5):419-36.
48. Acharya M M, Martirosian V, Chmielewski N N, Hanna N, Tran K K, Liao A C. et al. Stem cell transplantation reverses chemotherapy-induced cognitive dysfunction. Cancer Res. 2015; 75(4):676-86.
49. Spratling M W. Cortical region interactions and the functional role of apical dendrites. Behav Cogn Neurosci Rev. 2002; 1(3):219-28.
50. Phillips W A, Bachmann T, Storm J F. Apical Function in Neocortical Pyramidal Cells: A Common Pathway by Which General Anesthetics Can Affect Mental State. Front Neural Circuits. 2018; 12:50.
51. Thelen M, Rosen A, Nairn A C, Aderem A. Regulation by phosphorylation of reversible association of a myristoylated protein kinase C substrate with the plasma membrane. Nature. 1991; 351(6324):320-2.
52. Hains A B, Vu M A, Maciejewski P K, van Dyek C H, Gottron M, Amsten A F. Inhibition of protein kinase C signaling protects prefrontal cortex dendritic spines and cognition from the effects of chronic stress. Proc Natl Acad Sci USA. 2009; 106(42):17957-62.
53. Pourmohammadi N, Alimoradi H, Mehr S E, Hassanzadeh G, Hadian M R, Sharifzadeh M, et al. Lithium attenuates peripheral neuropathy induced by paclitaxel in rats. Basic Clin Pharmacol Toxicol. 2012; 110(3):231-7.
54. Nguyen L D, Ehrlich B E. Cellular mechanisms and treatments for chemobrain: insight from aging and neurodegenerative diseases. EMBO Mol Med. 2020:e12075.
55. Wang H Y, Friedman E. Lithium inhibition of protein kinase C activation-induced serotonin release. Psychopharmacology (Berl). 1989; 99(2):213-8.
56. Hahn C G, Umapathy, Wang H Y, Koneru R, Levinson D F, Friedman E. Lithium and valproic acid treatments reduce PKC activation and receptor-G protein coupling in platelets of bipolar manic patients. J Psychiatr Res. 2005; 39(4):355-63.
57. Saxena A, Scaini G, Bavaresco D V, Leite C, Valvassori S S, Carvalho A F, et al. Role of Protein Kinase C in Bipolar Disorder: A Review of the Current Literature. Mol Neuropsychiatry. 2017; 3(2):108-24.
58. Birnbaum S G, Yuan P X, Wang M, Vijayraghavan S, Bloom A K, Davis D J, et al. Protein kinase C overactivity impairs prefrontal cortical regulation of working memory. Science. 2004; 306(5697):882-4.
59. He Y, Wang Z J. Nociceptor beta II, delta, and epsilon isoforms of PKC differentially mediate paclitaxel-induced spontaneous and evoked pain. J Neurosci. 2015; 35(11):4614-25.
60. Brudvig J J, Weimer J M. X MARCKS the spot: myristoylated alanine-rich C kinase substrate in neuronal function and disease. Front Cell Neurosci. 2015; 9:407.
61. Chen C H, Cheng C T, Yuan Y, Zhai J, Arif M, Fong L W, et al. Elevated MARCKS phosphorylation contributes to unresponsiveness of breast cancer to paclitaxel treatment. Oncotarget. 2015; 6(17):15194-208.
62. LaBerge D. Sustained attention and apical dendrite activity in recurrent circuits. Brain Res Brain Res Rev. 2005; 50(1):86-99.
63. LaBerge D. Apical dendrite activity in cognition and consciousness. Conscious Cogn. 2006; 15(2):235-57.
64. Radley J J, Rocher A B, Janssen W G, Hof P R, McEwen B S, Morrison J H. Reversibility of apical dendritic retraction in the rat medial prefrontal cortex following repeated stress. Exp Neurol. 2005; 196(1):199-203.
65. Martin K P, Wellman C L. NMDA receptor blockade alters stress-induced dendritic remodeling in medial prefrontal cortex. Cereb Cortex. 2011; 21(10):2366-73.
66. Hains A B, Yabe Y, Arnsten A F. Chronic Stimulation of Alpha-2A-Adrenoceptors With Guanfacine Protects Rodent Prefrontal Cortex Dendritic Spines and Cognition From the Effects of Chronic Stress. Neurobiol Stress. 2015; 2:1-9.
67. Liston C, Miller M M, Goldwater D S, Radley J J, Rocher A B, Hof P R, et al. Stress-induced alterations in prefrontal cortical dendritic morphology predict selective impairments in perceptual attentional set-shifting. J Neurosci. 2006; 26(30):7870-4.
68. Alpar A, Ueberham U, Bruckner M K, Seeger G, Arendt T, Gartner U. Different dendrite and dendritic spine alterations in basal and apical arbors in mutant human amyloid precursor protein transgenic mice. Brain Res. 2006; 1099(1):189-98.
69. Shimoda A, Tsuzuki M, Keino H, Satoh M, Chiba Y, Saitoh Y, et al. Apical vulnerability to dendritic retraction in prefrontal neurones of ageing SAMP10 mouse: a model of cerebral degeneration. Neuropathol Appl Neurobiol. 2006; 32(1):1-14.

1. A method of reducing the likelihood of, inhibiting or reversing one or more symptoms of chemobrain in a patient or subject secondary to anticancer therapy with a chemotherapy agent which induces chemotherapy induced adverse effects in the central nervous system of said patient or subject comprising administering to the patient or subject an effective amount of a protein kinase c (PKC) inhibitor to said patient prior to, at the same time as or after the administration of said chemotherapy agent to said patient or subject.

2. The method according to claim 1 wherein said PKC inhibitor is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432, ruboxistaurin, miyabenol C, myricitrin, gossypol, verbascoside, BIM-1, staurosporine, sotrastaurine, bryostatin 1, Gouml 6983, Go 6976, Ro 32-0432, enzastaurin (LY317615), GSK690693, fasudil (JA-1077), mitoxantrone, bisindolylmaleimide (GF109203X), RO31-8220, rotterin, K252a, baicalein, quercetin, luteolin, bisindolylmaleimide II, calphostin C, L-threo-dihydrosphingosine, D-erythro-sphogosine, melittin, midostaurin (PKC412), CGP 533353, CRT 0066864, (±)-palmitoylcarnitine, PKC412, PKCβ pseudosubstrate, PKCζ pseudosubstrate, z-pseudosubstrate inhibitory peptide (ZIP) or a pharmaceutical salt or mixture thereof.

3. The method according to claim 1 or 2 wherein said PKC inhibitor is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432 hydrochloride salt or a pharmaceutical salt or mixture thereof.

4. The method according to any of claims 1-3 wherein said PKC inhibitor is chelerythrine or a pharmaceutically acceptable salt thereof.

5. The method according to any of claims 1-4 wherein said chemotherapy agent is a taxane, a vinca alkaloid, cyclophosphamide, doxorubicin, 5-Fluorouracil, a pharmaceutically acceptable salt or a mixture thereof.

6. The method according to claim 5 wherein said taxane is paclitaxel, docetaxel, IDN 5390, GRN1005 or a mixture thereof.

7. The method according to claim 5 or 6 wherein said taxane is paclitaxel or docetaxel or a mixture thereof.

8. The method according to any of claims 1-5 wherein said chemotherapeutic agent is a vinca alkaloid.

9. The method according to claim 8 wherein said vinca alkaloid is vinblastine, vincristine, vindesine, vinorelbine or a mixture thereof.

10. The method according to any of claims 1-4 wherein said chemotherapy agent is paclitaxel, docetaxel, vinblastine, vindesine, vinorelbine or a mixture thereof.

11. The method according to any of claims 1-4 said chemotherapy agent is cyclophosphamide, doxorubicin, 5-fluorouracil or a mixture thereof.

12. The method according to any of claims 1-11 wherein a lithium salt is also administered to said patient or subject.

13. The method according to any of claims 1-11 wherein a lithium salt s administered before, concurrently with or after said PKC inhibitor.

14. The method according to any of claims 1-11 wherein a lithium salt is administered before, concurrently with or after said chemotherapeutic agent.

15. The method according to any of claims 12-14 wherein said lithium salt is selected from the group consisting of lithium chloride, lithium carbonate, lithium acetate, lithium sulfate, lithium citrate, lithium orotate, lithium gluconate or a mixture thereof.

16. The method according to any of claims 12-15 wherein said lithium salt is lithium chloride, lithium carbonate or a mixture thereof.

17. The method according to claim 1 wherein said PKC inhibitor is cheleythrine and said anticancer agent is paclitaxel or docetaxel.

18. The method according to any one of claims 1-17 wherein said anticancer therapy is used to treat lung cancer, breast cancer, ovarian cancer and/or prostate cancer.

19. The method according to any of claims 1-18 wherein said anticancer therapy is used to treat breast or ovarian cancer.

20. The method according to any one of claims 1-17 wherein the cancer is breast, ovarian, prostate, cervical (especially during pregnancy), testicular, head and neck cancer, Hodgkin's lymphoma, non-small cell lung cancer, lymphoma, brain cancer, neuroblastoma, leukemia, cancer of the bladder, stomach, thyroid, soft tissue sarcoma, multiple myeloma, colon cancer, esophageal cancer, stomach cancer or pancreatic cancer.

21. The method according to any of claims 1-20 wherein said PKC inhibitor or said PKC inhibitor and said lithium salt are administered to said patient before the initiation of anticancer therapy.

22. The method according to any of claims 1-20 wherein said PKC inhibitor or said PKC inhibitor and said lithium salt are co-administered to said patient during anticancer therapy.

23. The method according to any of claims 1-20 wherein said PKC inhibitor or said PKC inhibitor and said lithium salt are administered to said patient after anticancer therapy.

24. A method of reducing the likelihood of, inhibiting or reversing one or more symptoms of chemobrain in a patient or subject secondary to anticancer therapy with a chemotherapy agent which induces chemotherapy induced adverse effects in the central nervous system of said patient or subject comprising administering to the patient or subject an effective amount of a protein kinase c (PKC) inhibitor to said patient prior to, at the same time as or after the administration of said chemotherapy agent to said patient or subject, wherein said chemotherapy agent is paclitaxel, doxetaxel, vinblastine, vincristine, vinorelbine, doxorubicin, cyclophosphamide or 5-fluorouracil, a pharmaceutically acceptable salt or mixture thereof and said PKC inhibitor is chelerythrine or a pharmaceutically acceptable salt thereof.

25. The method according to claim 24 wherein lithium carbonate or lithium chloride is administered concurrently with said PKC inhibitor.

26. A pharmaceutical composition comprising a combination of at least one PKC inhibitor and a lithium salt in effective amounts to reduce the likelihood of or inhibiting or reversing one or more symptoms of chemobrain in a patient or subject secondary to anticancer therapy with a chemotherapy agent which induces chemotherapy induced adverse effects in the central nervous system of said patient or subject.

27. The composition of claim 26 further including an anticancer effective amount of an anticancer compound selected from the group consisting of a taxane, a vinca alkaloid, cyclophosphamide, daunorubicin, 5-Fluorouracil or a mixture thereof.

28. The composition according to either of claims 26-27 wherein said PKC inhibitor is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432, ruboxistaurin, miyabenol C, myricitrin, gossypol, verbascoside, BIM-1, staurosporine, sotrastaurine, bryostatin 1, Gouml 6983, Go 6976, Ro 32-0432, enzastaurin (LY317615), GSK690693, fasudil (JA-1077), mitoxantrone, bisindolylmaleimide (GF109203X), RO31-8220, rotterin, K252a, baicalein, quercetin, luteolin, bisindolylmaleimide II, calphostin C, L-threo-dihydrosphingosine, D-erythro-sphogosine, melittin, midostaurin (PKC412), CGP 533353, CRT 0066864, (±)-palmitoylcarnitine, PKC412, PKCβ pseudosubstrate, pseudosubstrate, z-pseudosubstrate inhibitory peptide (ZIP) or a pharmaceutical salt or mixture thereof.

29. The composition according to any of claims 26-28 wherein said PKC inhibitor is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432 hydrochloride salt or a pharmaceutical salt or a mixture thereof.

30. The composition according to any of claims 27-29 wherein said taxane is paclitaxel, docetaxel, IDN 5390, GRN1005 or a mixture thereof.

31. The composition according to any one of claims 27-29 wherein said taxane is paclitaxel or docetaxel or a mixture thereof.

32. The composition according to any of claims 27-29 wherein said vinca alkaloid is vinblastine, vincristine, vindesine, vinorelbine or a mixture thereof.

33. The composition according to any of claims 27-29 wherein said chemotherapy agent is cyclophosphamide, doxorubicin, 5-fluorouracil or a mixture thereof.

34. The composition according to any of claims 26-33 wherein said lithium salt is selected from the group consisting of lithium chloride, lithium carbonate, lithium acetate, lithium sulfate, lithium citrate, lithium orotate, lithium gluconate or a mixture thereof.

35. The composition according to any of claims 26-34 wherein said lithium salt is lithium chloride, lithium carbonate or a mixture thereof.

36. A pharmaceutical composition comprising a combination of at least one PKC inhibitor and at least one chemotherapy agent which induces chemotherapy induced adverse effects in the central nervous system of a patient or subject, said PKC inhibitor being included in said composition in effective amounts to reduce the likelihood of or inhibiting or reversing one or more symptoms of chemobrain in said patient or subject secondary to anticancer therapy with said chemotherapy agent.

37. The composition of claim 36 wherein said chemotherapy agent is selected from the group consisting of a taxane, a vinca alkaloid, cyclophosphamide, daunorubicin, 5-Fluorouracil or a mixture thereof.

38. The composition according to claim 36 or 37 wherein said PKC inhibitor is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432, ruboxistaurin, miyabenol C, myricitrin, gossypol, verbascoside, BIM-1, staurosporine, sotrastaurine, bryostatin 1, Gouml 6983, Go 6976, Ro 32-0432, enzastaurin (LY317615), GSK690693, fasudil (JA-1077), mitoxantrone, bisindolylmaleimide (GF109203X), RO31-8220, rotterin, K252a, baicalein, quercetin, luteolin, bisindolylmaleimide II, calphostin C, L-threo-dihydrosphingosine, D-erythro-sphogosine, melittin, midostaurin (PKC412), CGP 533353, CRT 0066864, (±)-palmitoylcarnitine, PKC412, PKCβ pseudosubstrate, PKCζ pseudosubstrate, z-pseudosubstrate inhibitory peptide (ZIP) or a pharmaceutical salt or mixture thereof.

39. The composition according to any of claims 36-37 wherein said PKC inhibitor is chelerythrine, ruboxistaurin, miyabenol C, myricitrin, gossypol, verbascoside, BIM-1, staurosporine, sotrastaurine, bryostatin 1, Gouml 6983, Go 6976, Ro 32-0432 hydrochloride salt or a pharmaceutical salt or a mixture thereof.

40. The composition according to any of claims 37-39 wherein said taxane is paclitaxel, docetaxel, IDN 5390, GRN1005 or a mixture thereof.

41. The composition according to any one of claims 37-40 wherein said taxane is paclitaxel or docetaxel or a mixture thereof.

42. The composition according to any one of claims 37-39 wherein said vinca alkaloid is vinblastine, vincristine, vindesine, vinorelbine or a mixture thereof.

43. The composition according to any one of claims 37-39 wherein said chemotherapy agent is cyclophosphamide, doxorubicin, 5-fluorouracil or a mixture thereof.

44. The composition according to any of claims 37-43 further comprising an effective amount of a lithium salt selected from the group consisting of lithium chloride, lithium carbonate, lithium acetate, lithium sulfate, lithium citrate, lithium orotate, lithium gluconate or a mixture thereof.

45. The composition according to 44 wherein said lithium salt is lithium chloride, lithium carbonate or a mixture thereof.

46. The composition according to claim 44 or 45 wherein said lithium salt is lithium chloride.
